(19) United States
(12) Patent Application Publication
(10) Pub. No.: US 2026/0315460 A1
(43) Pub. Date: Oct. 15, 2026

(54) EXTRACELLULAR MATRIX MODULATING AGENT

(71) Applicants: Friedrich E. KRUSE, Erlangen (DE); Ursula SCHLOTZER-SCHREHARDT, Erlangen (DE); KOWA COMPANY, LTD., Nagoya-shi (JP)

(72) Inventors: Friedrich E. KRUSE, Erlangen (DE); Ursula SCHLOTZER-SCHREHARDT, Erlangen (DE)

(73) Assignees: Friedrich E. KRUSE, Erlangen (DE); Ursula SCHLOTZER-SCHREHARDT, Erlangen (DE); KOWA COMPANY, LTD., Nagoya-shi (JP)

(21) Appl. No.: 19/467,932

(22) Filed: Feb. 3, 2026

Related U.S. Application Data
(63) Continuation of application No. 17/430,068, filed on Feb. 27, 2020, now abandoned.

(30) Foreign Application Priority Data
Feb. 28, 2019 (WO) PCT/JP2019/007735
Sep. 30, 2019 (JP) 2019-179428

(51) Int. Cl. A61K 31/496; A61P 27/02

(52) U.S. Cl. CPC A61K 31/496; A61P 27/02

(57) ABSTRACT

The present invention provide an agent for modulating expression of extracellular matrix by corneal endothelial cells. The present invention pertains to an agent for modulating expression of extracellular matrix by corneal endothelial cells containing, 1-(4-fluoro-5-isoquinolinesulfonyl)-2-methyl-1,4-homopiperazine or a salt thereof, or a solvate thereof.

### TECHNICAL FIELD

The present invention relates to an agent for modulating expression of extracellular matrix by corneal endothelial cells. More specifically, the present invention relates to an agent for modulating expression of extracellular matrix by corneal endothelial cells containing, as an active ingredient, 1-(4-fluoro-5-isoquinolinesulfonyl)-2-methyl-1,4-homopiperazine or a salt thereof, or a solvate thereof.

### BACKGROUND ART

The cornea is an important tissue not only composing the wall of the eyeball with the sclera, but also behaving as an entrance to take in an image of the outside into the eye by virtue of a transparent tissue. The cornea has a thickness of approximately 500 μm at the central part, and consists of five layers of, starting from the outside, corneal epithelium, Bowman membrane, corneal stroma, Descemet's membrane, and corneal endothelium.

Corneal endothelial cells produce various extracellular matrix components to form a specialized basement membrane, i.e. Descemet's membrane. Descemet's membrane is composed of two layers, an anterior banded layer (fetal layer) and a posterior nonbanded layer (postnatal) that continuously grows and thickens throughout life by the secretory activity of the endothelial cells. A normal Descemet′ membrane contains collagen type VIII, collagen type IV (chains α1-α2), and fibronectin on its stromal side and entactin, laminin, perlecan, and collagen type IV (chains α3-α6) on its endothelial side. Virtually all corneal endothelial disorders, such as pseudophakic bullous keratopathy, are associated with abnormal extracellular matrix accumulation. In these conditions, corneal endothelial cells undergo stress-induced trans-differentiation into myofibroblasts, which produce excessive amounts of collagens (mostly collagen type I) forming abnormal posterior fibrillary layers on the surface of Descemet's membrane. Such a posterior fibrotic layer is supposed to be the result of a common final pathway in endothelial dysfunction (see Non-Patent Document 1).

An altered matrix composition of Descemet's membrane together with the formation of wart-like excrescences, so-called guttae, also represent a characteristic and early clinical hallmark of Fuchs endothelial corneal dystrophy (FECD) (FIG. 1). These matrix alterations lead to loss of contrast sensitivity and increased glare in the affected patients due to light scattering (see Non-Patent Document 2), thereby severely impacting on the patients' quality of vision. Corneal endothelial cell dysfunction further results in corneal edema, loss of corneal transparency, and irreversible blindness. FECD is a slowly progressive bilateral corneal disease that becomes clinically evident in adults aged over 40. In 2015, FECD was responsible for 39% of corneal grafts performed worldwide. There are currently no treatments for FECD other than corneal transplantation.

In a recent publication, it has shown that FECD is characterized by a specific change in expression patterns of matrix proteins, which differ from expression patterns of patients with pseudophakic bullous keratopathy and normal subjects (see Non-Patent Document 3). Several matrix proteins to be selectively upregulated in FECD specimens, including collagen type I, III and XVI, fibronectin, agrin, laminin α1, TGFBI and clusterin, both on the mRNA and protein level were identified. Immunohistochemistry confirmed the participation of these matrix proteins, particularly of collagen type III, in Descemet thickening and guttae formation (FIG. 2). Abnormal matrix deposition in FECD may be induced by increased levels of TGF-β2 in the aqueous humor of FECD patients. Upregulation of collagen type VI, laminin α3, and fibronectin was recently confirmed in endothelial cell cultures derived from FECD patients (see Non-Patent Document 4).

Knowing that Descemet thickening and guttae formations are the first clinical signs of FECD, inhibition of abnormal matrix deposition could slow down the progression of the disease and eventually avoid corneal transplantation. Inventors of the present invention generated the hypothesis that stress-or TGFβ-induced Rho kinase (Rho-associated, coiled-coil containing protein kinase: ROCK) signaling activation is involved in the fibrotic response and abnormal matrix production by corneal endothelial cells and that ROCK inhibition can attenuate, prevent or even reverse these alterations, thereby normalizing visual function in FECD patients.

Rho is a small GTPase, which upon activation by guanine nucleotide exchange factors activates ROCK that phosphorylates various substrates, including myosin light chain and LIM kinase. ROCK signaling is activated by wounding, integrin stimulation, cytokines and growth factors and regulates a wide spectrum of fundamental cellular events, including adhesion, migration, proliferation, differentiation and apoptosis. These processes are mainly mediated via modulation of the cytoskeleton.

A body of evidence has been accumulated with regard to the involvement of the Rho-ROCK signaling pathway in the development of fibrotic lesions in multiple organ systems including the kidney and lungs (see Non-Patent Document 5-8). ROCK signaling activation appears to be involved in pro-fibrotic responses of epithelial, endothelial and mesenchymal cells in response to stress or injury, driving the transition of fibroblasts into myofibroblasts producing an altered collagen-rich extracellular matrix. Consistently, ROCK inhibitors have been used to suppress or prevent fibrosis in animal models, and more importantly, induce the regression of already established fibrosis (see Non-Patent Document 6). These anti-fibrotic effects may be mediated by preventing the TGFβ-induced myofibroblast transformation and excessive matrix production (see Non-Patent Document 9-12). Fasudil, a small molecule inhibitor of ROCK, has been shown to have antifibrotic effects in various fibrotic diseases, e.g. it attenuated the expression of α-SMA, MLCP, LIMK1, p-cofilin, collagen I, and collagen III protein in human fibroblasts (see Non-Patent Document 12). Multiple lines of evidence therefore indicate that ROCK inhibition has great potential to be a powerful therapeutic tool in the treatment of fibrosis.

The transforming growth factor-β (TGF-β) signaling pathway is a key mediator of fibroblast activation that drives the aberrant synthesis of extracellular matrix in fibrotic diseases. Moreover, activation of canonical Wnt signaling is necessary for TGF-β-mediated fibrosis and highlights a key role for the interaction of both pathways in the pathogenesis of fibrotic diseases (see Non-Patent Document 13). Wnt and TGF-β signaling have been also suggested to activate endothelial-to-mesenchymal transition (EMT).

Previous studies showed that inhibition of ROCK signaling using Y-27632, H-1152 and Thiazovivin stimulated cell adhesion, migration, proliferation and wound healing in vitro and in vivo animal models and suppressed apoptosis and endothelial-mesenchymal transition (EMT) of corneal endothelial cells (see Non-Patent Document 14-25). Similar findings have been reported for K-115 stimulating endothelial cell proliferation and wound healing, upregulation of functional endothelial markers and downregulation of EMT markers (see Non-Patent Document 26 and 27). These studies provided evidence for the usefulness of ROCK inhibitors for corneal endothelial cell cultivation, as adjunct drugs in cell-injection therapies (see Non-Patent Document 28), and as eye drops for rescue strategies in unsuccessful descemetorhexis (descemet's membrane removing) without cornea donner transplantation, which procedure is so called DWEK (Descemetorhexis without endothelial keratoplasty) or DSO (Descemet's stripping only), for FECD (see Non-Patent Document 29).

### CITATION LIST

### Non Patent Literature

Non Patent Literature 1: Ljubimov A. et al., Invest. Ophthalmol. Vis. Sci., 1996, 37, 997-1007

Non Patent Literature 2: Watanabe S. et al., Ophthalmology, 2015, 122(10), 2013-2019

Non Patent Literature 3: Weller J. et al., Invest. Ophthalmol. Vis. Sci., 2014, 55, 3700-3708

Non Patent Literature 4: Goyer B. et al., Tissue Engineering, 2018, 24(7&8), 607-615

Non-Patent Document 5: Moriyama T. and Nagatoya K., Drug News Perspect., 2004, 17(1), 29-34

Non-Patent Document 6: Knipe R. et al., Pharmacol. Rev., 2015, 67, 103-117

Non-Patent Document 7: Riches D. et al., Am. J. Pathol., 2015, 185(4), 909-912

Non-Patent Document 8: Shimizu T. and Liao J., Circ. J., 2016, 80, 1491-1498

Non-Patent Document 9: Zhu J. et al., Int. J. Ophthalmol., 2013, 6(1), 8-14

Non-Patent Document 10: Gu L. et al., Chem. Pharm. Bull., 2013, 61(7), 688-694

Non-Patent Document 11: Baba I. et al., Mol. Med. Rep., 2015, 12, 8010-8020

Non-Patent Document 12: Xu N. et al., Am. J. Trans. Res., 2017, 9(3), 1317-1325

Non-Patent Document 13: Akhmetshina A. et al., Nat. Commun., 2012, 3, 735

Non-Patent Document 14: Okumura N. et al., Invest. Ophthalmol. Vis. Sci., 2009, 50, 3680-3687

Non-Patent Document 15: Okumura N. et al., Br. J. Ophthalmol., 2011, 95, 1006-1009

Non-Patent Document 16: Okumura N. et al., Invest. Ophthalmol. Vis. Sci., 2013, 54, 2439-2502

Non-Patent Document 17: Okumura N. et al., Invest. Ophthalmol. Vis. Sci., 2014, 55(1), 318-329

Non-Patent Document 18: Okumura N. et al., Invest. Ophthalmol. Vis. Sci., 2015, 56, 6067-6074

Non-Patent Document 19: Okumura N. et al., Scientific Reports, 2016, 6, 26113

Non-Patent Document 20: Pipparelli A. et al., PLoS One, 2013, 8(4), E62095

Non-Patent Document 21: Li S. et al., Tissue Cell, 2013, 45(6), 387-396

Non-Patent Document 22: Guo Y. et al., Cellular Reprogramming, 2015, 17(1), 77-87

Non-Patent Document 23: Peh G. et al., Scientific Reports, 2015, 5, 9167

Non-Patent Document 24: Meekins L. et al., Invest. Ophthalmol. Vis. Sci., 2016, 57, 6731-6738

Non-Patent Document 25: Wu Q. et al., Int. J. Mol. Med., 2017, 40, 1009-1018

Non-Patent Document 26: Nakagawa H. et al., PLoS One, 2015, 10(9), e0136802

Non-Patent Document 27: Okumura N. et al., Invest. Ophthalmol. Vis. Sci., 2016, 57, 1284-1292

Non-Patent Document 28: Kinoshita S. et al., New Eng. J. Med., 2018, 378(11), 995-1003

Non-Patent Document 29: Moloney G. et al., Cornea, 2017, 36(6), 642-648

### SUMMARY OF INVENTION

### Problems to be Solved by the Invention

The present invention provides an agent for modulating expression of extracellular matrix by corneal endothelial cells, and an agent for suppressing the guttae formation in FECD patient.

### Means for Solving the Problems

As a result of intensive studies carried out by the inventors in order to solve the above-mentioned problems, it is found that 1-(4-fluoro-5-isoquinolinesulfonyl)-2-methyl-1,4-homopiperazine or a salt thereof or a solvate thereof is capable of suppressing the expression of extracellular matrix by corneal endothelial cells, such as agrin, collagen types I and III, and fibronectin, and results in a medical agent for treatment of corneal diseases characterized by abnormal matrix production. Moreover, 1-(4-fluoro-5-isoquinolinesulfonyl)-2-methyl-1,4-homopiperazine or a salt thereof or a solvate thereof is capable of modulating the TGF-beta signaling pathway and the transformation of myofibroblast, and results in a medical agent for treatment of fibrosis in patients with early stages of FECD.

In addition, it is found that it is also possible to instill the substance in the eye, enabling its formulation which imposes only a slight burden on a patient.

In other words, the present invention relates to an agent for modulating the expression of extracellular matrix by corneal endothelial cells comprising 1-(4-fluoro-5-isoquinolinesulfonyl)-2-methyl-1,4-homopiperazine or a salt thereof, or a solvate thereof.

In addition, the present invention relates to an agent for preventing and/or suppressing the guttae formation comprising 1-(4-fluoro-5-isoquinolinesulfonyl)-2-methyl-1,4-homopiperazine or a salt thereof, or a solvate thereof.

Furthermore, the present invention relates to an agent for preventing and/or suppressing the recurrence of the guttae formation comprising 1-(4-fluoro-5-isoquinolinesulfonyl)-2-methyl-1,4-homopiperazine or a salt thereof, or a solvate thereof.

A more detailed description of the present invention is as follows.
(1) An agent for modulating the expression of extracellular matrix by corneal endothelial cells comprising 1-(4-fluoro-5-isoquinolinesulfonyl)-2-methyl-1,4-homopiperazine or a salt thereof, or a solvate thereof.
(2) The agent for modulating the expression of extracellular matrix by corneal endothelial cells of above-mentioned (1), wherein the 1-(4-fluoro-5-isoquinolinesulfonyl)-2-methyl-1,4-homopiperazine is (S)-(-)-1-(4-fluoro-5-isoquinolinesulfonyl)-2-methyl-1,4-homopiperazine.
(3) The agent for modulating the expression of extracellular matrix by corneal endothelial cells of above-mentioned (1) or (2), wherein the agent for modulating the expression of extracellular matrix in corneal endothelial cells is an agent for preventing and/or suppressing the guttae formation.
(4) The agent for preventing and/or suppressing the guttae formation of above-mentioned (3), wherein the agent for preventing and/or suppressing the guttae formation is the agent for preventing and/or suppressing the guttae formation in FECD patient.
(5) The agent for preventing and/or suppressing the guttae formation in an FECD patient of above-mentioned (4), wherein the agent for preventing and/or suppressing the guttae formation in FECD patient is the agent for preventing and/or suppressing the guttae formation in early stage of FECD patient.
(6) The agent for modulating the expression of extracellular matrix by corneal endothelial cells of above-mentioned (1) or (2), wherein the agent for modulating the expression of extracellular matrix by corneal endothelial cells is an agent for preventing and/or suppressing the recurrence of the guttae formation.
(7) The agent for preventing and/or suppressing the recurrence of the guttae formation of above-mentioned (6), wherein the agent for preventing and/or suppressing the recurrence of the guttae formation is the agent for preventing and/or suppressing the recurrence of the guttae formation in FECD patient.
(8) The agent for preventing and/or suppressing the recurrence of the guttae formation in FECD patient of above-mentioned (7), wherein the agent for preventing and/or suppressing the recurrence of the guttae formation in FECD patient is the agent for preventing and/or suppressing the recurrence of the guttae formation in FECD patient after descemetorhexis without cornea donner transplantation (DWEK or DSO).
(9) The agent any one of above-mentioned (1) to (8), wherein the agent is a liquid formulation.
(10) The agent for modulating the expression of extracellular matrix by corneal endothelial cells of above-mentioned (1) or (2), wherein the agent for modulating the expression of extracellular matrix by corneal endothelial cells is an agent for preventing and/or treating disorder of corneal endothelium.
(11) The agent for modulating the expression of extracellular matrix by corneal endothelial cells of above-mentioned (10), wherein the disorder of corneal endothelium is a disease of corneal endothelium such as bullous keratopathy or corneal endotheliitis.
(12) The agent for modulating the expression of extracellular matrix by corneal endothelial cells of any one of above-mentioned (1) to (11), wherein the agent for modulating the expression of extracellular matrix by corneal endothelial cells is an eyedrop.
(13) A method for producing a formulation of the agent for modulating the expression of extracellular matrix by corneal endothelial cells of any one of above-mentioned (1) to (12), comprising: mixing 1-(4-fluoro-5-isoquinolinesulfonyl)-2-methyl-1,4-homopiperazine or a salt thereof, or a solvate thereof with a pharmaceutically acceptable carrier.
(14) An agent for modulating the TGF-beta signaling pathway and the transformation of myofibroblast in patients with early stages of FECD comprising 1-(4-fluoro-5-isoquinolinesulfonyl)-2-methyl-1,4-homopiperazine or a salt thereof, or a solvate thereof.
(15) An agent for modulating the TGF-beta signaling pathway in patients with early stages of FECD comprising 1-(4-fluoro-5-isoquinolinesulfonyl)-2-methyl-1,4-homopiperazine or a salt thereof, or a solvate thereof.
(16) An agent for modulating the transformation of myofibroblast in patients with early stages of FECD comprising 1-(4-fluoro-5-isoquinolinesulfonyl)-2-methyl-1,4-homopiperazine or a salt thereof, or a solvate thereof.
(17) The agent any one of above-mentioned (14) to (16), wherein the agent is for treatment of fibrosis in patients with early stages of FECD.
(18) The agent any one of above-mentioned (14) to (17), wherein the agent is a liquid formulation.

### Effects of the Invention

The present invention provides an agent for modulating expression of extracellular matrix by corneal endothelial cells in order to prevent and/or treat corneal disorder causing by the abnormality of the expression of extracellular matrix by corneal endothelial cells. The agent for modulating expression of extracellular matrix by corneal endothelial cells of the present invention may prevent and/or treat various kinds of disorders of the corneal endothelium, for example, a disease of the corneal endothelium such as bullous keratopathy or corneal endotheliitis, or the abnormalities of the corneal endothelium caused by corneal transplant or the like. Moreover, the agent for modulating expression of extracellular matrix by corneal endothelial cells of the present invention is effective even though the active ingredient thereof is at low concentrations, therefore the agent for modulating expression of extracellular matrix by corneal endothelial cells of the present invention may be used as a pharmaceutical composition which is highly effective and safe with few side effects.

In addition, the agent for modulating expression of extracellular matrix by corneal endothelial cells of the present invention may provide an eyedrop imposing only a slight burden on a patient.

### BRIEF DESCRIPTION OF DRAWINGS

FIG. 1 illustrates Schematic diagram of the structure of Descemet's membrane in healthy controls and FECD patients.

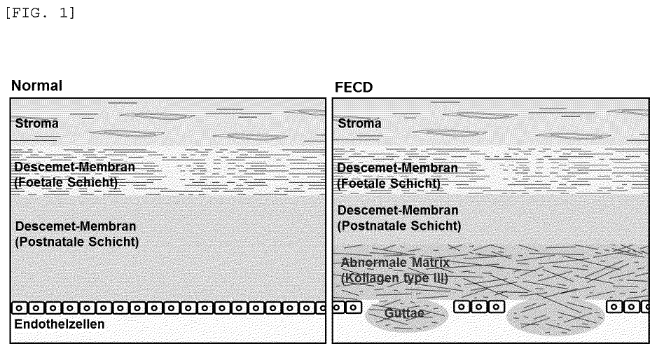

FIG. 2 illustrates Immunohistochemical staining of collagen type III in Descemet's membrane and in guttae of FECD patients.

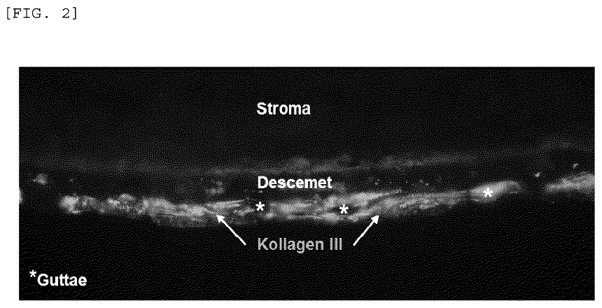

FIG. 3 illustrates Relative expression of FECD-associated matrix genes in corneal endothelial cells of normal donors (n=6) without and with incubation in 10-100 μM K-115 for 24 h. Gene expression was analyzed by quantitative real-time PCR and normalized to GAPDH (*p<0.01).

FIG. 4 illustrates Relative expression of FECD-associated matrix genes in corneal endothelial cells of FECD patients (n=20) without and with incubation in 30 μM K-115 for 24 h. Gene expression was analyzed by quantitative real-time PCR and normalized to GAPDH (*p<0.01, **p<0.001).

FIG. 5 illustrates Western Blot analysis of fibronectin in corneal endothelial cells of FECD patients (n=4); equal loading of samples was verified by β-actin. Densitometric analysis of band intensities shows mean values ±SD of 4 independent experiments (*p<0.01).

### MODES FOR CARRYING OUT THE INVENTION

Hereinafter, a description is made of the present invention in more detail.

1-(4-Fluoro-5-isoquinolinesulfonyl)-2-methyl-1,4-homopiperazine in the present invention has one asymmetric carbon atom, leading to an (R) isomer and (S) isomer. In the present invention, any of the (R) isomer, (S) isomer, and a mixture thereof may be used. As the pharmaceutical active ingredient, a highly-pure optically-active material of the (R) isomer or (S) isomer is preferable. From the terms of the desired activity, the (S) isomer is the more preferable than the (R) isomer.

1-(4-Fluoro-5-isoquinolinesulfonyl)-2-methyl-1,4-homopiperazine, which is an active ingredient of the present invention, is publicly known as a compound having a Rho kinase inhibitory effect, which may be produced by a publicly known method, e.g., the method disclosed in WO 99/20620 A1. The (S) isomer of 1-(4-Fluoro-5-isoquinolinesulfonyl)-2-methyl-1,4-homopiperazine is also known as K-115 or Ripasudil, which is manufactured and sold as a therapeutic agent for glaucoma and ocular hypertension. Glanatec (trade name; 0.4% Ripasudil hydrochloride hydrate) ophthalmic solution as an eye drop formulation is clinically available in Japan.

A salt of 1-(4-Fluoro-5-isoquinolinesulfonyl)-2-methyl-1,4-homopiperazine includes, for example, a salt formed with an inorganic acid such as hydrochloric acid, sulfuric acid, nitric acid, hydrofluoric acid or hydrobromic acid, or a salt formed with organic acid such as acetic acid, tartaric acid, lactic acid, citric acid, fumaric acid, maleic acid, succinic acid, methanesulfonic acid, ethanesulfonic acid, benzenesulphonic acid, toluenesulfonic acid, naphthalenesulfonic acid or camphorsulfonic acid. In particular, a hydrochloride salt is preferable.

1-(4-Fluoro-5-isoquinolinesulfonyl)-2-methyl-1,4-homopiperazine or a salt thereof may exist not only as an unsolvated type but also as a hydrate or solvate. Although a hydrate is preferable, the present invention includes all crystal forms and hydrates or solvates.

As a Rho kinase inhibitor, AR-13324 (chemical name: 4-[(2S)-3-amino-1-(6-isoquinolinylamino)-1-oxo-2-propanyl]benzyl 2,4-dimethylbenzoate; Netarsudil (trade name); Rhopressa (trademark)) can also be used in this invention.

In the present invention, an “agent for modulating the expression of extracellular matrix by corneal endothelial cells” refers to those for modulating the expression of extracellular matrix by corneal endothelial cells to suppress the increased function of the corneal endothelial cells in morbid condition, such as FECD.

Since the guttae formation in early or late stage of FECD patient mainly depends on the increased expression of extracellular matrix, such as agrin, collagen types I and III, and fibronectin, or the like in corneal endothelial cells, the “agent for modulating the expression of extracellular matrix by corneal endothelial cells” of the present invention is considered to be capable of preventing or suppressing the progress of FECD.

Because administering the “agent for modulating the expression of extracellular matrix by corneal endothelial cells” of the present invention makes it possible to modulate the function of the corneal endothelium, the “agent for modulating the expression of extracellular matrix by corneal endothelial cells” of the present invention may also be used as an agent for preventing or treating corneal edema, and/or an agent for preventing or treating disorder of the corneal endothelium.

The “modulating the expression of extracellular matrix by corneal endothelial cells” of the present invention is formulated into a dosage form suitable for topical administration to the eye using a usual formulation technology frequently used in the art. As the dosage form, for example, a liquid formulation such as, but is not limited to, an injection for anterior chamber, ocular perfusion, or eyedrop is preferable. As the preferable formulation, from the terms of the therapeutic effect, an injection for anterior chamber or ocular perfusion is a preferable dosage form, but imposes a significant burden on a patient. Accordingly, from the terms of easy administration, the preferable formulation includes an eyedrop.

Preparation of the eyedrop may be achieved by, for example, dissolving or suspending the desired above-mentioned component in an aqueous solvent such as sterilized pure water or saline, or a nonaqueous solvent such as vegetable oil including cottonseed oil, soy oil, sesame oil or peanut oil, adjusting the solution or suspension pressure to a predetermined osmotic pressure, and performing sterilization treatment such as filtration sterilization. Note that when preparing an ophthalmic ointment, an ointment base may be contained in addition to the above-mentioned various components. The said ointment base preferably includes, but not particularly limited to; an oleaginous base such as vaseline, liquid paraffin or polyethylene; an emulsion base in which the oil phase and aqueous phase are emulsified with a surfactant or the like; a water-soluble base consisting of hydroxypropylmethylcellulose, carboxymethylcellulose, polyethyleneglycol, or the like.

When using 1-(4-fluoro-5-isoquinolinesulfonyl)-2-methyl-1,4-homopiperazine, preferably (S)-(-)-1-(4-fluoro-5-isoquinolinesulfonyl)-2-methyl-1,4-homopiperazine or a salt thereof, or a solvate thereof for the “modulating the expression of extracellular matrix by corneal endothelial cells” of the present invention, the dose depends on the body weight, age, sex, symptom of a patient, the dosage form, the number of doses and the like, but generally, the dose of 1-(4-fluoro-5-isoquinolinesulfonyl)-2-methyl-1,4-homopiperazine, preferably (S)-(-)-1-(4-fluoro-5-isoquinolinesulfonyl)-2-methyl-1,4-homopiperazine for an adult includes a range of 0.025-10000 μg a day, preferably 0.025-2000 μg, more preferably 0.1-2000 μg, further 0.025-200 μg, 0.025-100 μg.

When using the same as an eyedrop, the concentration of the active ingredient may be approximately 0.0001-5 w/v %, preferably approximately 0.01-4 w/v %.

In addition, the number of doses is not limited in particular, but the administration is preferably performed one or several times, and in case of the liquid eyedrop, one to several drops may be instilled in the eye for once administration.

### EXAMPLES

Endothelial-Descemet membrane (EDM)-complexes from normal donor corneas and from FECD patients during DMEK surgery were prepared. EDM scrolls were dissected into two halves and incubated for 24-72 hours in CorneaMax storage medium (Eurobio) with or without K-115 (Selleck Chemicals). Gene expression analysis was performed using specific real-time PCR arrays for FECD-associated candidate genes as well as RT2 Profiler PCR Arrays (Quiagen). Array results were again validated using gene-specific real-time PCR assays. Protein expression analysis was performed by Western blotting.

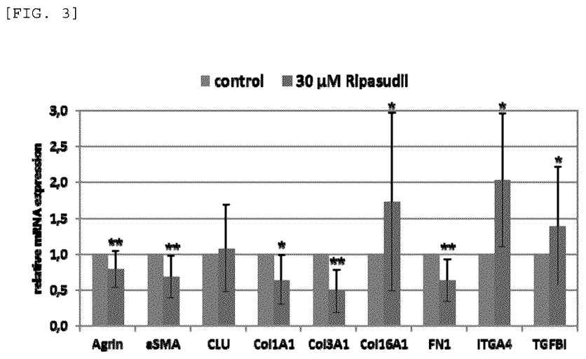

First, expression levels of those matrix genes were analyzed, which were pathologically upregulated in endothelial cells from FECD patients, in normal EDM specimens using various concentrations of K-115. It was found that the majority of matrix proteins (agrin, collagen types I and III, fibronectin) as well as α-smooth muscle actin (α-SMA) and β-actin were significantly downregulated and that a concentration of 30 μM K-115 was most effective (FIG. 3).

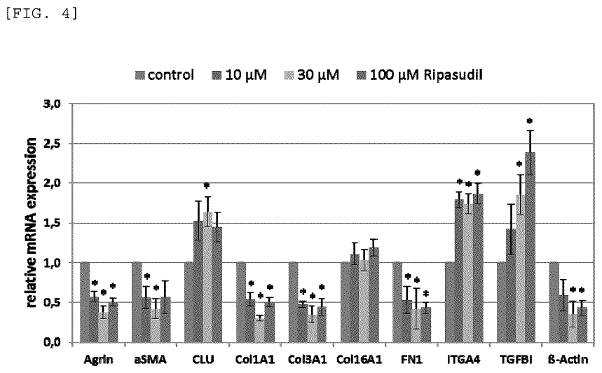

Then, gene expression levels in FECD specimens were analyzed, which were incubated in 30 μM K-115 for 24 hours. A significant reduction in expression levels of agrin, collagen types I and III, fibronectin and also α-SMA upon K-115 treatment compared to untreated controls (FIG. 4) were observed. Collagen type XVI, integrin α4 (ITGA4) and TGFBI were upregulated, but these proteins are not involved in guttae formation, but are mainly involved in cell-matrix interaction and cell adhesion.

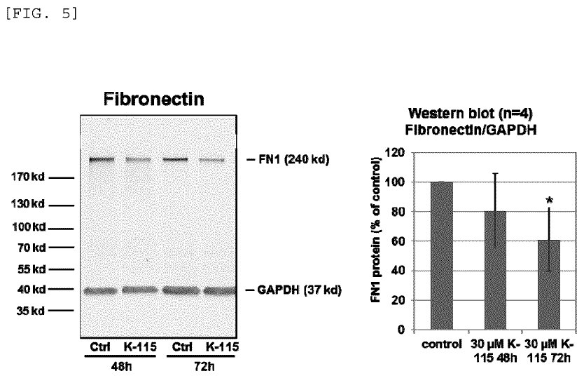

Selected candidate genes, such as fibronectin and collagen type III, were also tested on the protein level and were confirmed to be significantly downregulated in FECD specimens upon treatment with 30 μM K-115 after 72 hours of incubation (FIG. 5).

In order to identify additional matrix genes that may be transcriptionally regulated by K-115, gene expression profiling using a Human Extracellular Matrix and Adhesion Molecule PCR array were performed. With this approach, downregulation of fibronectin and collagen types I and III and detected additional downregulation of vitronectin and collagen types V and XIV in corneal endothelial cells from FECD patients (data not shown) were confirmed.

In essence, these data support the notion that ROCK inhibition using K-115 positively influences abnormal corneal endothelial matrix metabolism by downregulating the mRNA and protein expression of FECD-associated matrix genes contributing to Descemet thickening and guttae formation. K-115 is, therefore, suggested as a new treatment modality and anti-fibrotic strategy for corneal endothelial cell “rejuvenation” in FECD and other corneal endothelial diseases.

1. A method for downregulating the expression of an extracellular matrix by corneal endothelial cells in a subject, comprising:
administering to the subject 1-(4-fluoro-5-isoquinolinesulfonyl)-2-methyl-1,4-homopiperazine, a salt thereof, or a solvate thereof.

2. The method according to claim 1, wherein the administering comprises administering (S)-(-)-1-(4-fluoro-5-isoquinolinesulfonyl)-2-methyl-1,4-homopiperazine, a salt thereof, or a solvate thereof.

3. A method for preventing and/or suppressing guttae formation in a subject, comprising:
administering to the subject 1-(4-fluoro-5-isoquinolinesulfonyl)-2-methyl-1,4-homopiperazine, a salt thereof, or a solvate thereof.

4. The method according to claim 3, wherein the subject is an FECD patient.

5. A method for preventing and/or suppressing recurrence of guttae formation in a subject, comprising:
administering to the subject 1-(4-fluoro-5-isoquinolinesulfonyl)-2-methyl-1,4-homopiperazine, a salt thereof, or a solvate thereof.

6. The method according to claim 5, wherein the subject is an FECD patient.

7. The method according to claim 1, wherein the administering comprises administering a liquid formulation comprising 1-(4-fluoro-5-isoquinolinesulfonyl)-2-methyl-1,4-homopiperazine, a salt thereof, or a solvate thereof.

8. The method according to claim 7, wherein the liquid formulation comprises (S)-(-)-1-(4-fluoro-5-isoquinolinesulfonyl)-2-methyl-1,4-homopiperazine, a salt thereof, or a solvate thereof.

9. The method according to claim 2, wherein the administering comprises administering from 0.025 to 10000 μg a day of (S)-(-)-1-(4-fluoro-5-isoquinolinesulfonyl)-2-methyl-1,4-homopiperazine.

10. The method according to claim 2, wherein the administering comprises administering from 0.025 to 100 μg a day of (S)-(-)-1-(4-fluoro-5-isoquinolinesulfonyl)-2-methyl-1,4-homopiperazine.

11. The method according to claim 2, wherein the administering comprises administering an eyedrop comprising from 0.0001 to 5 w/v % of (S)-(-)-1-(4-fluoro-5-isoquinolinesulfonyl)-2-methyl-1,4-homopiperazine.

12. The method according to claim 2, wherein the administering comprises administering an eyedrop comprising from 0.01 to 4 w/v % of (S)-(-)-1-(4-fluoro-5-isoquinolinesulfonyl)-2-methyl-1,4-homopiperazine.

13. The method according to claim 3, wherein the administering comprises administering a liquid formulation comprising 1-(4-fluoro-5-isoquinolinesulfonyl)-2-methyl-1,4-homopiperazine, a salt thereof, or a solvate thereof.

14. The method according to claim 13, wherein the liquid formulation comprises (S)-(-)-1-(4-fluoro-5-isoquinolinesulfonyl)-2-methyl-1,4-homopiperazine, a salt thereof, or a solvate thereof.

15. The method according to claim 4, wherein the administering comprises administering a liquid formulation comprising 1-(4-fluoro-5-isoquinolinesulfonyl)-2-methyl-1,4-homopiperazine, a salt thereof, or a solvate thereof.

16. The method according to claim 15, wherein the liquid formulation comprises (S)-(-)-1-(4-fluoro-5-isoquinolinesulfonyl)-2-methyl-1,4-homopiperazine, a salt thereof, or a solvate thereof.

17. The method according to claim 5, wherein the administering comprises administering a liquid formulation comprising 1-(4-fluoro-5-isoquinolinesulfonyl)-2-methyl-1,4-homopiperazine, a salt thereof, or a solvate thereof.

18. The method according to claim 17, wherein the liquid formulation comprises (S)-(-)-1-(4-fluoro-5-isoquinolinesulfonyl)-2-methyl-1,4-homopiperazine, a salt thereof, or a solvate thereof.

19. The method according to claim 6, wherein the administering comprises administering a liquid formulation comprising 1-(4-fluoro-5-isoquinolinesulfonyl)-2-methyl-1,4-homopiperazine, a salt thereof, or a solvate thereof.

20. The method according to claim 19, wherein the liquid formulation comprises (S)-(-)-1-(4-fluoro-5-isoquinolinesulfonyl)-2-methyl-1,4-homopiperazine, a salt thereof, or a solvate thereof.
